(19) United States
(12) Patent Application Publication
(10) Pub. No.: US 2013/0137962 A1
(43) Pub. Date: May 30, 2013

(54) Method for Detecting Arthritis and Cartilage Damage Using Magnetic Resonance Sequences

(71) Applicants: Kenneth L. Urish, Hershey, PA (US); Matthew G. Keffalas, Woburn, MA (US); Timothy J. Mosher, Elizabethtown, PA (US); David J. Miller, State College, PA (US)

(72) Inventors: Kenneth L. Urish, Hershey, PA (US); Matthew G. Keffalas, Woburn, MA (US); Timothy J. Mosher, Elizabethtown, PA (US); David J. Miller, State College, PA (US)

(21) Appl. No.: 13/691,359

(22) Filed: Nov. 30, 2012

Related U.S. Application Data
(60) Provisional application No. 61/629,876, filed on Nov. 30, 2011.

(51) Int. Cl. A61B 5/055

(52) U.S. Cl. CPC A61B 5/055
USPC 600/410

(57) ABSTRACT

In this work, a Magnetic Resonance Imaging (MRI)-based automatic classifier was designed to predict changes due to osteoarthritis (OA) years prior to their symptomatic presentation and radiographic detection. For each patient, multiple image texture features were measured from the T2 map of the patella cartilage and the lateral and medial compartments of the femoral condyle. A support vector machine (SVM)-based linear discriminant function was trained to predict health status, as well as the affected knee compartment. Feature selection was integrated into the classifier training to drastically reduce the number of image (biomarker) features without sacrificing classification accuracy. It was found that a dominant knee compartment determined the classification decision for most patients. We demonstrate that the signal texture index (STI) predicts disease progression prior to symptoms or radiographic signs of OA. In symptomatic individuals, the STI correlates with the pain and severity of OA suggesting it is a sensitive measure of the same on T2 Maps. These observed changes localized to one knee compartment demonstrating the method can localize OA to specific regions.

### CROSS-REFERENCE TO RELATED APPLICATION

This application is the perfection of U.S. Provisional Application Ser. No. 61/629,876, filed on Nov. 30, 2011, the disclosure of which is fully incorporated by reference herein.

### BACKGROUND OF THE INVENTION

1. Field of the Invention

The present invention relates to using magnetic resonance imaging signal metrics, including but not limited to texture metrics, for prognosticating and diagnostic measurements of osteoarthritis (“OA”) and cartilage damage.

2. Relevant Art

Numerous references are known using MRI data to some extent in the same context as arthritis or cartilage. Such references include: Mangialaio et al PCT Application No. 2006/008183 (pertaining to the use of biomarkers for rheumatoid arthritis (or “RA”)); Licha et al. EP Application No. 1,931,391 (which employed optical imaging for RA); Wakitani et al. EP Application No. 2,128,615 (for detecting joint cartilage damage); Bukowski et al. PCT Application No. 2009/135219 (for detecting a predisposition for osteoarthritis); and Yen et al. Published U.S. Patent Application No. 2012/0128593 (for using MRI imaging in inflammation or infection detection). The most recent, Dam et al. U.S. Pat. No. 8,300,910 segmented cartilage and mentioned homogeneity but only in the context of damaged or suspect joints.

This invention represents an alternative to and improvement over the foregoing. With respect to Dam et al., this concept is distinct in our using a large number of several different MR signal measurements and combine same into a single measurement by selecting only the important, more critical measurements for assessing not only damaged joints but also those joints currently showing no sign of disease or damage for prognostication and imaging biomarkers.

Osteoarthritis (OA) is a common disease affecting approximately half of the population above the age of 55. Radiographs remain the standard imaging technique to assess OA disease progression. There is an interest in using Magnetic Resonance Imaging (MRI) to identify pre-radiographic changes in OA.

MRI has the capability to directly image cartilage. There have been preliminary applications of compositional MRI techniques to detect changes in water and proteoglycan content and anisotropy of collagen fibers [footnoted reference nos. 1-4 below] associated with early degradation, albeit with limited success [fn. ref. 5]. The inclusion of cartilage T2 mappings in the Osteoarthritis Initiative (OAI) protocol was designed in part to develop such predictive capability. This more than 4-year longitudinal natural history study provides annual knee MRI examinations of almost 5000 subjects, and is a valuable resource for deriving image based biomarkers to identify individuals at risk for incident OA or rapid OA progression. The study provides the longitudinal data necessary for evaluation of cartilage T2 as a potential biomarker for predicting OA progression.

Normal T2 values of knee articular cartilage have a well-recognized pattern of signal variation, spatial signal distribution that changes with OA. The T2 values of articular cartilage are strongly dependent on the orientation of the type II collagen matrix with respect to orientation of the applied magnetic field BO (anisotropy) [fn. ref nos. 2, 6], and regional differences in cartilage water content [fn. ref nos. 2, 5, 7]. In normal cartilage, regional variation in the collagen fiber anisotropy and water content produces variation in the pattern of cartilage T2 values. This structural organization provides a well-recognized pattern of signal variation in MRI T2-weighted images, where low signal is observed near bone, gradually increasing in signal intensity toward the articular surface.

We postulated that disruption of this signal variation may be an early change of OA before the presence of symptoms or radiographic changes. Loss of collagen matrix anisotropy, one of the earliest processes in OA [fn. ref. 8], leads to focal elevation in cartilage water content, increased mobility of the extra-cellular water, and ultimately loss of the ability of cartilage to with stand repetitive compressive loading. While early degeneration of the collagen matrix produces an elevation in cartilage T2, further degradation of cartilage produces heterogeneity in T2 values, with regions of cartilage demonstrating foci of low T2 values [fn. ref 9].

Recently, other groups have demonstrated a change in texture metrics in populations of patients with increased OA risk factors supporting this idea. Increased heterogeneity in the spatial distribution of cartilage T2 values is also a characteristic of aging, likely reflecting senescent degradation of the collagen matrix [fn. ref nos. 2, 10]. Because T2 can increase or decrease regionally in cartilage, the bulk T2 value, which represents an average of multiple cartilage voxels over a region of interest (ROI), may remain unchanged, even while the variation of cartilage T2 from voxel to voxel may increase substantially. There may be no simple image “signature” of the disease that can be easily visualized and interpreted. Evidence of early OA progression in cartilage may manifest by subtle changes in image texture that occur on multiple scales across the huge space of voxels in the T2 map. The use of automated statistical classification techniques is directly motivated by problems of this nature where the data is high-dimensional. Together, these suggest that evaluation of changes in the pattern of cartilage T2 with OA progression may be a more responsive and reliable measure of cartilage degeneration than the change in absolute T2 values.

### SUMMARY OF THE INVENTION

In this work, an MRI-based automatic classifier is designed to predict changes due to OA years prior to both their symptomatic presentation and radiographic detection. 220 patients were selected from the Osteoarthritis Initiative (OAI) database, 89 healthy and 131 symptomatic, based on the change in total WOMAC score from baseline to three year follow-up. For each patient, at baseline, 725 image texture features were measured from the T2 map of the patella cartilage and the lateral and medial compartments of the femoral condyle. A support vector machine (SVM)-based linear discriminant function was trained to predict health status, as well as the affected knee compartment, at three years from baseline. Feature selection was integrated into the classifier training to drastically reduce the number of image (biomarker) features without sacrificing classification accuracy. When the most important 20 of these 725 image features are used the method achieved an accuracy of 80% with a sensitivity of 79.2% and specificity of 68.5%. Further, it was found that a dominant knee compartment determined the classification decision for most patients. With this method, one may localize and identify regions of arthritis and cartilage damage to the patient's joint.

We demonstrate that the signal texture index (STI) predicts disease progression prior to symptoms or radiographic signs of OA, and, in symptomatic individuals, the STI correlate with the pain and severity of osteoarthritis suggesting it is a sensitive measure of early OA on T2 Maps. Further, these observed changes localized to one knee compartment suggesting that early OA occurs in primarily one compartment. Additional studies are required to determine whether the STI can be used to predict disease progression after post traumatic OA in the knee or in different joints and demonstrate response to therapeutic progression. The proposed method has clinical application for early arthritis diagnosis and treatment, the development and study of surgical procedures for cartilage repair and preservation, and to help identify and follow study populations to support both epidemiological and drug studies.

We hypothesized that this regional signal heterogeneity on T2 maps can be used as an early imaging biomarker to predict OA progression in asymptomatic individuals and as sensitive measure of early signs of OA. Early degenerative changes in the structural organization and water content of collagen in OA would be expected to have a regional change in signal as measured on T2 maps. These changes in regional heterogeneity can be quantified by texture metrics. We have utilized the OAI to define a population of individuals with no symptoms or radiographic sings of OA that are known to have rapid symptomatic progression in three years and a comparison asymptomatic control population. Image features were extracted from both populations and compared using classification to quantify and compare signal heterogeneity. We demonstrate that signal texture index can predict OA progression prior to OA onset. Further, the texture image features that are associated with OA progression are localized in a dominant compartment that is highly correlated with the mechanical axis of the knee. Our approach is to effectively utilize the signal texture index as a marker of cartilage degeneration, quantitatively assessed by measured texture features.

### BRIEF DESCRIPTION OF DRAWINGS

Further features, objectives and advantages of this invention will be made clearer with the following detailed description made with reference to the accompanying drawings in which:

FIG. 1 is a schematic flowchart according to one embodiment of this invention;

FIG. 2A is a graph comparing signal texture index (STI) versus density as means for identifying early signs of OA on a T2 map;

FIG. 2B is a graph plotting trial data using an SVM classifier from twenty dimensions onto two dimensions;

FIG. 3 is a graph plotting true versus false positive rates with the invention, the diagonal line therein representing random guessing;

FIG. 4A is a graph plotting STI versus density with the first, second and third compartments indicated along with the SVM decision boundary;

FIG. 4B is a graph showing the average STI, by notched box plots, for each of the three compartments; and

FIG. 4C is a graph plotting for individuals with a dominant medial or lateral compartment, the varus or valgus mechanical axis alignment associated with an increased STI.

### DESCRIPTION OF PREFERRED EMBODIMENTS

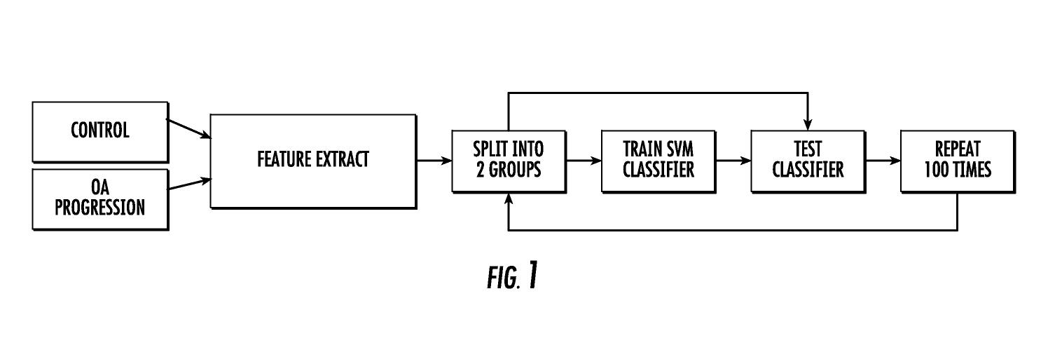

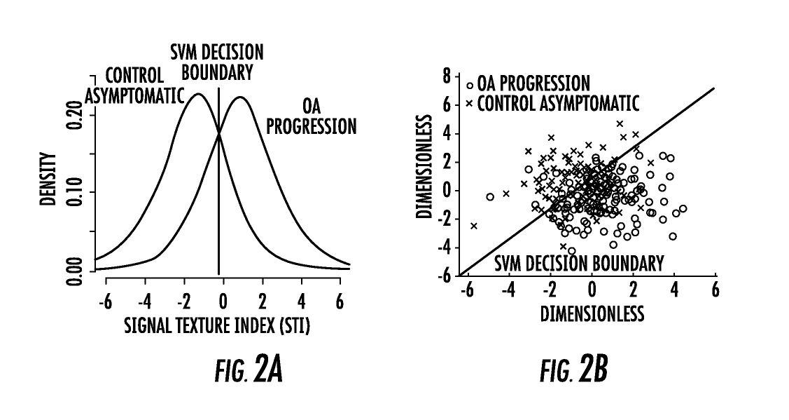

First, referring to the accompanying drawings, FIG. 1 shows a schematic representation of one experimental design according to this invention. The Signal texture index identifies early signs of OA on T2 maps per accompanying FIG. 2. More particularly, FIG. 2A plotted histograms of the Signal Texture Index (STI) for the control and OA populations. A positive score therein corresponded to an OA decision, and a negative score a control decision. Accuracy was about 80%. Therein, the SVM decision boundary is indicated by the solid vertical line to the left of “0”. The results shown are the combined 1000 trials with an average number of 20 features needed to build the STI.

For FIG. 2B visualization, multidimensional scaling was used to project the data of one such trial using the SVM classifier from twenty dimensions onto two dimensions. Admittedly, that has some cost in representation fidelity. The axes are dimensionless, and represent a summation of the different image features used to determine the signal texture index (or “STI”).

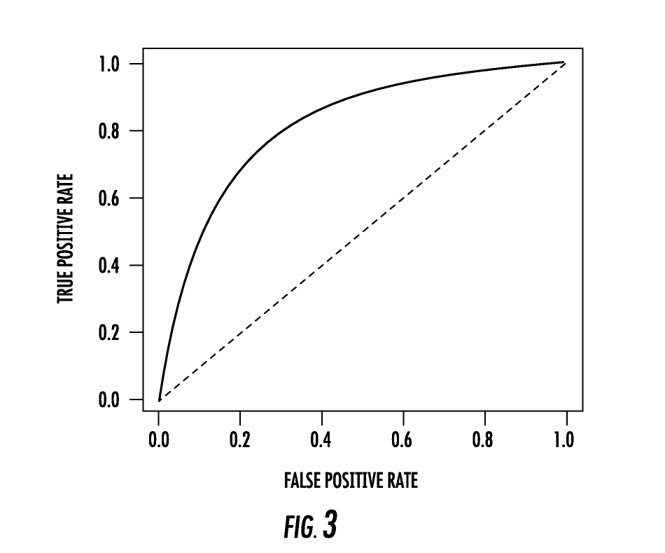

In FIG. 3, Receiver operating characteristic (or “ROC”) curve—The sensitivity is equivalent to the true positive rate, and specificity equivalent to true negative rate. The diagonal line therein represents the results of random guessing.

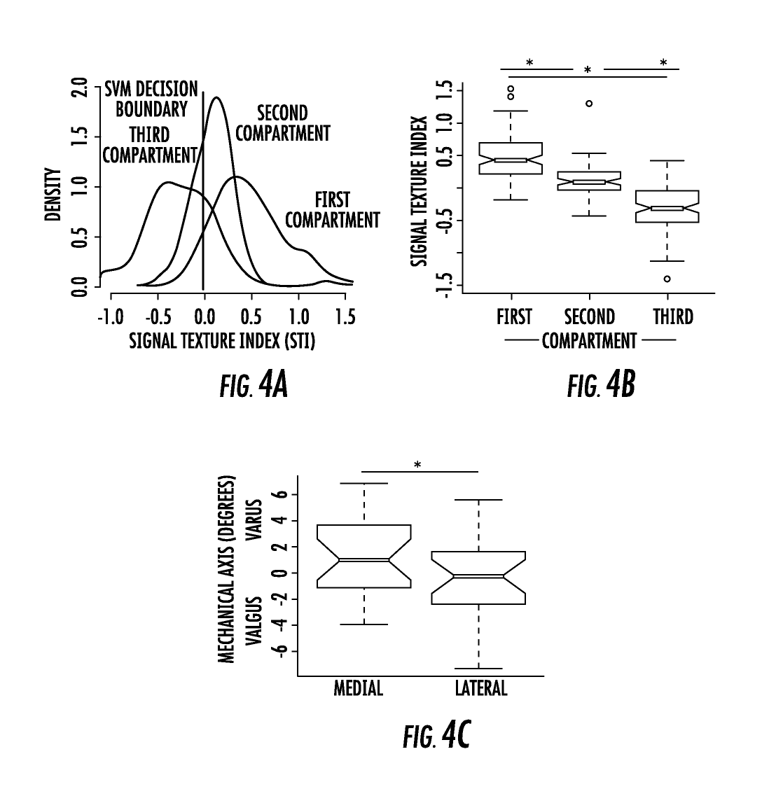

For the charts at FIG. 4, a signal texture index (STI) that indicates OA is associated primarily with one knee compartment. More particularly, FIG. 4A showed how the features that dominate OA decisions, for most subjects, come from primarily one knee compartment (medial, lateral, or patella). To demonstrate this, the aggregate STI “partial scores” were calculated for each knee compartment in each subject. A histogram for the compartment with largest partial score (“First”), second largest partial score (“Second”), and minimum partial score (labeled “Third”) across subjects as a function of Density where the area under each curve is unity. In that same FIG. 4A, the SVM decision score is shown as the vertical black line.

For FIG. 4B, the average STI in each compartment of 4A is statistically different. Notched box plots show the average STI for each of the three compartments. Finally, in FIG. 4C, the dominant compartment that predicted OA progression is strongly correlated with mechanical alignment. Notched box plot of individuals with a dominant medial or lateral compartment contribution to STI was compared as a function of the mechanical axis. Individuals with a varus alignment were associated with an increased STI in the medial compartment and vice versa for valgus alignment. The patella as the dominant compartment in the texture index was excluded. Notches note a 95% confidence interval of the mean. Negative mechanical axis values indicate valgus alignment. * p<0.05.

Population Cohort: Patients were selected from the OAI cohort. A total of 201 patients were selected, 89 control and 112 symptomatic. Specific inclusion criteria are as follows. Control subjects were selected from the control cohort defined as a low WOMAC score (<5) with low KL score that had no risk factors for OA progression. The OA rapid progression cohort were selected from the incidence cohort based on the initial criteria of a low WOMAC pain score less than 10 that had no radiographic signs of OA (KL<1) and that had a change in WOMAC pain score of >10. The incidence cohort did not have OA risk factors or symptomatic OA (risk factors: 1. previous knee surgery; 2. overweight as defined by ages cutoffs of 45-69 males>92.9 kg and females >77.1 kg; 3. previous knee injury defined by an injury of difficulty walking for at least one week; 4. family history in parent of sibling of total knee replacement; 5. Heberden's Nodes defined as self-report of bony enlargement of one or more enlargements of the distal interphalangeal joints in either hand; symptomatic OA: 1. Kellgren and Lawrence (KL) grade <2 on fixed flexion radiographs; 2. no frequent knee symptoms for at least one month during the past 12 months defined as “pain, aching, or stiffness in or around the knee on most days”). The incidence cohort did have risk factors for OA progression (OAI exclusion criteria included rheumatoid arthritis, bilateral total knee joint replacement, and a positive pregnancy test. Institutional review board approval had been obtained at all participating institutions in the OAI, and informed consent had been obtained by all participants in the study.

MR Image Acquisition: In the OAI cohort, three dimensional sagital DESS and T2 mapping images were acquired from the imaging database freely available by request [11]. Briefly, MRI of the knee joint was performed on a 3.0 T Siemens whole body MAGNETOM Trio 3T scanner (Siemens, Erlangen, Germany) using a standard extremity coil. For high-spatial-resolution 3D DESS imaging [fn. ref. 12], a total of 160 sections were acquired with a field of view (FOV) of 14 cm (matrix 384×384) with an in-plane spatial resolution of 0.365×0.365 mm and a slice thickness of 0.7 mm with an acquisition time of 11 min. For sagittal 2D dual-echo fast spin echo (FSE) sequence for mapping T2 relaxation time, TR was 2700 ms and 7 echo images with TE ranging 10-80 ms were acquired with matrix of 384×384, in-plane resolution of 0.313×0.313 mm, FOV of 12 cm, acquisition time 12 min and slice thickness of 3 mm. OAI data sets used included the baseline imaging data set 0.E.1 and 0.C.2.

Plain Radiographic Assessment: Standard bilateral standing posterior-anterior fixed flexion knee radiographs were obtained at the baseline visit. Knees were positioned with a 20°-30° flexion and 10° internal rotation of the feet in a plexiglass frame (SynaFlexer, CCBR-Synarc, San Francisco, Calif., USA). Knee radiographs were graded using the using the Kellgren-Lawrence (KL) scoring system (Lawrence, 1957). The patello-femoral joint was not included in the KL score as the OAI protocol used the fixed flexion knee radiograph for KL scoring.

Standard bilateral, full length lower limb radiographs were obtained at the one-year clinical visit with knees fully extended and feet place six inches apart directly facing the film centered at the knees. Mechanical axis was measured using the standard technique of measuring the angle placed from the center of the femoral head to the medial tibial prominence to the midline of the ankle (McGory 2002, pub med id:12439260). OAI data sets used included the baseline and one year imaging data set 0.E.1 and 0.C.2.

Clinical Assessment: Clinical symptoms were assessed with the Western Ontario and McMaster Universities Osteoarthritis (WOMAC) questionnaire at the time of magnetic resonance screening (Bellamy, id 3068365). The OAI clinical data set 0.2.2 was used for data collection.

Registration: DESS and T2 images were registered using the Mattes mutual information metric. Registration software was built using the insight toolkit, a C++ open source image analysis library (www.itk.org). DESS images possess higher resolution and were transformed through three-dimensional space to preserve the voxel information on the fixed T2 image using a verser transform. Linear interpolation was used in sampling voxels on non-grid positions. A specialized gradient decent optimizer is used to define the transform parameters through successive iterations as the search space is large across 6 degrees of freedom. After the transform, the mutual information metric is used to assess the degree of alignment between the two images and the process is repeated until a maximum degree of overlap has been achieved.

Segmentation: Segmentation was completed on DESS images. Segmentation of the femoral and patellar cartilage was completed using custom semi-automated software implementing a global active statistical shape model with a local active contour model. Gross inaccuracies in the segmentation could be corrected by a manual correction of the computer segmentation. Binary masks of the lateral and medial femoral condyle and patella were generated from the segmented images. The lateral and medial masks were split into 5 sections for each individual. The patella region was treated as a single section. There were 11 regions of interest (ROI) per individual. This is an arbitrary number and any number of regions of interest could be selected in operation of this invention. The specific segmentation technique used is not significant for our method and any type of segmentation method selected from the group of automatic, semi-automatic, and manual may be used.

T2 Maps: T2 maps were calculated from the Multi-Slice-Multi-Echo T2 images available in the OAI. Calculation of the T2 maps have been previously described (Smith and Mosher; Pubmed id 11436214). Briefly, the T2 maps are calculated on a voxel-by-voxel basis using a linear least squares fitting with CCHIPS/IDL software (Cincinnati Children's Hospital Image Processing Software/Interactive Data Language, (RSI, Boulder, Colo.). The MR T2 signal decay of cartilage is mono-exponential, and the signal intensity decay can be expressed as an exponential decay as a function of time for each voxel. Quantitative T2 maps can be visualized as a color-coded image using an ordinal rainbow scale.

Image Feature Extraction: Candidate features were calculated from each T2 map using the segmented binary masks region of interest using a matlab script (Mathworks, Natick, Mass.). Each feature was independently measured in each of the 11 sections on each knee. There were four main categories of features: histogram, grey level co-occurrence matrix (GLCM), grey level run length matrix (GLRL), and z-score. The numbers reported below are the totals from all 11 sections. A 32-bin histogram was used to calculate the mean, variance, entropy, and central moments. GLCM features were calculated from the grey level co-occurrence matrices at unit distance and angles 0, 45, 90, 135 degrees, and 90 degrees in the z direction. GLRL features were calculated from grey level run length matrices at angles 0 and 90 degrees. The Z-score was calculated for all voxels in each section. The mean value, variance, minimum value, maximum value, and range of values were then calculated (n=55). In each of the 11 sections, a total of 725 features were measured on each T2 map. All features were normalized to the range [−1,1].

Classification, Feature Elimination, and Partial Sum Measurements: Support vector machine (SVM) training and testing were implemented using the LIBSVM Matlab interface. To assess the performance of the classifier, we randomly divided the entire cohort into 1000 equal-sized training and test subsets with equal numbers of control and rapid progression individuals. In each of the 1000 trials, the SVM classifier was trained to discriminate between control and rapid progression OA populations using all 725 features on the training set, and the accuracy of the classifier was measured on the independent test set. The confusion matrix was calculated after each trial. Margin based feature elimination (MFE) was used to eliminate redundant and uninformative candidate features. In the same trial, SVM training was coupled with MFE to identify a reduced set of essential features. The accuracy of the reduced feature set was tested on the test data set, and the confusion matrix was again determined (FIG. 1). After classification was completed and the signal texture index was calculated, the signal texture index of each compartment was determined. In each of the 100 trials, the partial weighted linear sum of the medial femoral condyle, lateral femoral condyle, and patella contribution to the each individuals overall SVM score was determined. Results were normalized based on the number of regions in each compartment (5 for the medial and lateral condyle, one for the patella), and averaged across the 100 separate trials.

Statistics: Data is expressed as a mean±standard deviation, except were noted. Direct comparisons between two cell populations were made using an unpaired, two-tailed Student's t-test. Statistical significance was determined if P<0.05. Multiple group comparison's were made using two-way ANOVA, using the Student-Newman-Keuls pairwise comparison to determine significance levels. Conventions for box plot include the mean outlined by the box representing the 25% and 75% quantiles, whiskers representing the minimum and maximum value, outliers denoted with a circle, and notches representing the 95% confidence interval of the mean.

Receiver operating characteristic (ROC) analysis was performed on the entire set using standard techniques. The procedure and methods are discussed in detail using the thesis of Matthew Keffalas, The Pennsylvania State University, Electrical Engineering, Schreyer Honors College, 2010.

We hypothesized that T2 map signal heterogeneity could accurately prognosticate OA progression. To test this hypothesis, we used the OAI to identify and compare the texture metrics of two populations of T2 maps: an asymptomatic control and a rapid OA progression population. The asymptomatic group was collected from the OAI control cohort (n=89). The rapid progression population was collected from the incidence cohort (n=112). At the initial time point, the population was asymptomatic (WOMAC <10) and had no radiographic signs of OA (KL ≦2). At the 3 year time point, this population experienced a WOMAC change (greater than 10), signifying both a large and rapid progression of symptoms. These populations were comparable in regards to age, sex, and BMI. As expected by cohort definitions between the control and incidence cohorts, the asymptomatic population did have lower WOMAC and KL scores than the rapidly progression population.

To assess signal heterogeneity, we quantified signal heterogeneity using a series of texture metrics on each of these populations. Asymptomatic and rapid progression populations based on baseline T2 map image features that described texture. Images were segmented and registered so that image texture features could be extracted. DESS images were used for segmentation because of the increased contrast at cartilage-soft tissue and cartilage-bone interfaces. Multimodality registration was used to align DESS and T2 sequences so that the segmentation masks (subdivided into ROI) could be used to measure a range of histogram and texture based image features. We chose as candidate features some well-known descriptors of image texture (local entropy, variance, cross-correlation, run-lengths, histogram based) and integrated a feature reduction step within the classifier training. An image classifier, SVM, was used to develop a model to predict OA, with classifier training and testing via a series of cross-validation experiments, dividing the subpopulations into training and test subsets. Margin based feature elimination was used to eliminate redundant and uninformative features, sacrificing minimal accuracy in order to simplify the model and to identify image feature biomarkers. The classifier design “wraps” MFE around SVM training, removing one feature at a time. The classifier found an appropriate hyper-plane in image-feature space that separated these populations. The SVM score is the distance from this plane, and can be described as a signal texture metric (FIG. 1).

Three separate cases of classifier accuracy were analyzed. First, the accuracy of using the entire set of all 725 features before feature elimination was measured. The average accuracy of the classifier was greater than 80%, corresponding to an average sensitivity of 82.0±5.4% and an average specificity of 75.0±7.3%. Second, MFE was used to remove redundant and uninformative features significantly reducing the feature space. An average of only 20 of the 725 features was needed to maintain a comparable level of accuracy. The average accuracy of the system with MFE feature selection was 76.1±7.2%, with average sensitivity of 79.1±6.7% and average specificity of 70.0±7.7%. Finally, in each of these trials by design, a new separate set of features was selected. If feature reduction was performed on the entire trial set simultaneously, a single set of features for the signal texture index were defined. At a sacrifice of some bias to obtain the single feature set, accuracy was 80% with a sensitivity of 83% and a specificity of 77%. The remainder of the discussion will focus on the second case as it presents the most unbiased classifier accuracy and quantification of signal texture index.

After feature reduction, the STI had good separation of the asymptomatic and OA populations. By design, the classifier sets a STI value of zero as the decision boundary so that any positive value is determined to be OA progression and any negative value a control decision is made (FIG. 2A). One of the trials with similar accuracy (76.6%) to the entire set of trial corresponds with good separation of the two populations (FIG. 2B). ROC analysis showed excellent classifier performance, and tradeoffs between specificity and sensitivity as a function of the SVM decision boundary (FIG. 3). This invention can be used to prognosticate and/or diagnose cartilage damage, arthritis, or the general state of cartilage health. This demonstrates the minimum accuracy of the invention. Small modifications will improve the accuracy.

The image texture features that predict rapid progression of OA for most individuals are primarily located in one of the three knee compartments. The STI is calculated from a weighted sum of image feature measurements from the lateral and medial compartment and the patella. By separately considering the features from each compartment (lateral, medial, patella) and finding the partial sum for each section, the effective contribution from each compartment to the overall decision can be determined. The rapid progression population was considered separately in this analysis. The contribution of features in each compartment to the overall signal texture index shows substantial separation between each compartment (FIG. 4A) and the mean of each of these compartments are statistically different (FIG. 4B). This suggests that, for most subjects, a single knee compartment plays a dominant role in rapid progression to symptomatic OA.

To test the observation that the signal texture index from one compartment plays a dominant role in OA progression, we isolated the medial and lateral sub-populations from the dominant compartment and compared the compartment to the mechanical axis from standing full limb length radiographs. Individuals with a dominant compartment on the medial condyle were highly correlated with valgus alignment, and individuals associated with a dominant compartment on the lateral condyle were associated with a varus alignment. A comparison of these two populations demonstrated the differences were statistically different as measured by the student's t-test. At a minimum, the dominant compartment's location is highly correlated with mechanical axis (FIG. 4C).

For symptomatic patients that are correctly classified, the most positive partial sum amongst the three sections contributes most to the correct decision. It can thus be inferred that the section with this partial sum is likely the one undergoing the most OA changes. Moreover, a significant disparity between the largest and second largest partial sums for an individual patient suggests OA changes may only be occurring in the knee section with the largest partial sum. Thus, this invention may be used to localize cartilage damage, the progression of disease, or identify area of the joint where healthy cartilage resides.

This invention had been demonstrated to be used on T2 maps for OA symptomatic prognostication but the demonstration is equally valid on a number of other instances. Any MR sequence including but not limited to dGERMIC (i.e, delayed gadolinium-enhanced magnetic resonance imaging of cartilage), T1, T1 rho, and T2 sequences could be substituted or combined with T2 maps. Texture and feature analysis was used to prognosticate but could be used as a diagnostic test for symptomatic progression or differentiate different stages of disease progression. Further, the same invention can be applied to predicting morphologic changes in articular cartilage including but not limited to changes in cartilage volume, area, or thickness and bone, synovial, inflammatory tissue morphometry. The invention is not specific to the disease of OA but any type of pathologic process effecting human or animal cartilage including but not limited to rheumatoid arthritis, post-traumatic arthritis, cartilage trauma, osteochondritis dissecans, or the general state of cartilage health. Also, the invention can be applied to any joint in the body including but not limited to hip, shoulder, elbow, wrist, and ankle The specific steps used in this invention can be altered in length, method employed, order, or omission.

A challenge in classification is the “curse of dimensionality”. There is a relative paucity of available training samples compared to the large dimensionality of the image feature space and to the number of parameters in the classifier model. This implies that the classifier has a tendency to overfit the data which can degrade the accuracy of the model. To avoid this problem, we applied a linear discriminant function classifier, SVM, to maximize the margin between these two populations. In this sense, the SVM maximizes the separation of the two classes. For an SVM, unlike a standard linear discriminate function, the number of model parameters is bounded by the number of training samples, rather than being controlled by the feature dimensionality. Since the number of samples is typically the much smaller number, in this way the SVM mitigates potential overfitting. SVM is not a unique solution to this process. Any linear or non-linear classifier and method of feature reduction can be applied for use in this invention. An emphasis was placed on defining OA by symptomatic progression. OA could be defined by symptoms, biomarkers, imaging criteria, or any other definition.

In addition to overcoming the problem of the non-linear response of T2 to cartilage degradation, this approach removes systematic bias. Differences in methodology or instrumentation used in the T2 measurement can lead to differences in the magnitude of T2 values. Since texture analysis compares spatial differences in T2 values between neighboring pixels rather than the absolute T2 values, it effectively uses an internal calibration standard to remove systematic bias. This helps eliminate the variation observed in a sequence as a function of the operator, machine, and location.

### FOOTNOTED REFERENCES ABOVE

1. David-Vaudey, E., et al., T2 relaxation time measurements in osteoarthritis. Magn Reson Imaging, 2004. 22(5): p. 673-82.
2. Mosher, T. J. and B. J. Dardzinski, Cartilage MRI T2 relaxation time mapping: overview and applications. Semin Musculoskelet Radiol, 2004. 8(4): p. 355-68.
3. Mosher, T. J., et al., MR imaging and T2 mapping of femoral cartilage: in vivo determination of the magic angle effect. AJR Am J Roentgenol, 2001. 177(3): p. 665-9.
4. Wheaton, A. J., et al., Correlation of T1 rho with fixed charge density in cartilage. J Magn Reson Imaging, 2004. 20(3): p. 519-25.
5. Burstein, D. and M. L. Gray, Is MRI fulfilling its promise for molecular imaging of cartilage in arthritis? Osteoarthritis Cartilage, 2006. 14(11): p. 1087-90.
6. Xia, Y., J. B. Moody, and H. Alhadlaq, Orientational dependence of T2 relaxation in articular cartilage: A microscopic MRI (microMRI) study. Magn Reson Med, 2002. 48(3): p. 460-9.
7. Harrison, M. M., et al., Patterns of knee arthrosis and patellar subluxation. Clin Orthop Relat Res, 1994(309): p. 56-63.
8. Maroudas, A. I., Balance between swelling pressure and collagen tension in normal and degenerate cartilage. Nature, 1976. 260(5554): p. 808-9.
9. Yulish, B. S., et al., Juvenile rheumatoid arthritis: assessment with MR imaging. Radiology, 1987. 165(1): p. 149-52.
10. Mosher, T. J., B. J. Dardzinski, and M. B. Smith, Human articular cartilage: influence of aging and early symptomatic degeneration on the spatial variation of T2 preliminary findings at 3 T. Radiology, 2000. 214(1): p. 259-66.
11. Peterfy, C. G., E. Schneider, and M. Nevitt, The osteoarthritis initiative: report on the design rationale for the magnetic resonance imaging protocol for the knee. Osteoarthritis Cartilage, 2008. 16(12): p. 1433-41.
12. Eckstein, F., et al., Double echo steady state magnetic resonance imaging of knee articular cartilage at 3 Tesla: a pilot study for the Osteoarthritis Initiative. Ann Rheum Dis, 2006. 65(4): p. 433-41.

What is claimed is:

1. A method for detecting a type of arthritis or damage to a joint or cartilage of a human patient comprises:
(a) taking a plurality of magnetic resonance (MR) image signal features from MR sequences from one or more regions of cartilage in a joint of the patient where arthritis or damage vulnerability is suspected;
(b) submitting said plurality of MR image signal features to a classifier for performing feature reduction that calculates which redundant or unnecessary features may be removed without materially impacting total feature accuracy;
(c) eliminating said redundant or unnecessary features from said plurality of MR image signal features to form a minimum grouping of image features for the patient;
(d) calculating from said minimum grouping of image features a signal texture index (STI) value; and
(e) comparing the STI value for the patient against at least two population databases, a first database including individuals known to develop arthritis or cartilage damage at a first time point and a second database including individuals known not to develop arthritis at a later time point.

2. The method of claim 1 which can be used to prognosticate, from the STI value, a likelihood of the patient developing arthritis or damage.

3. The method of claim 2 which can be used to prognosticate the likelihood that arthritis or damage will progress or regress in the patient.

4. The method of claim 3 which can be used to prognosticate the rate of progression or regression of arthritis in the patient.

5. The method of claim 1 wherein the arthritis to be detected is selected from the group consisting of osteoarthritis, rheumatoid arthritis, traumatic arthritis and cartilage degeneration.

6. The method of claim 1 wherein the damage to be detected is from a body area selected from the group consisting of the patient's knee, ankle, hip, shoulder, elbow, wrist or spine.

7. The method of claim 6 wherein the joint or cartilage damage to be detected is from the patient's knee, and the magnetic resonance (MR) sequences for that knee are taken for the patient's patella, medial and lateral compartments.

8. The method of claim 7 wherein the image features used to calculate the STI are taken mostly from a dominant compartment of the patient's knee where a majority of cartilage damage has occurred, said dominant compartment selected from the patient's patella, medial or lateral compartment.

9. The method of claim 1 wherein the classifier from step (b) is selected from the group consisting of a linear classifier, a non-linear classifier, a regression framework and a neural network.

10. The method of claim 1 wherein step (d) includes using one or more histogram measures selected from the group consisting of: average, mean, standard deviation, variance, dispersion, average energy, energy, skewness and kurtosis.

11. The method of claim 1 wherein step (d) includes using one or more measures selected from the group consisting of: gray level co-occurrence matrix (GLCM), gray level run length (GLRL), Z-scores, and general texture measurements.

12. A method for detecting a type of arthritis or other damage to cartilage of a patient's spine, or knee, ankle, hip, shoulder, elbow or wrist joint, said method comprising:
(a) taking a plurality of magnetic resonance (MR) image signal features from MR sequences from one or more regions of cartilage where arthritis or cartilage damage vulnerability is suspected;
(b) submitting said plurality of MR image signal features to a classifier for performing a feature reduction that calculates which features may be removed without materially impacting total feature accuracy;
(c) eliminating said features from said plurality of MR image signal features to form a minimum grouping of image features for the patient;
(d) calculating from said minimum grouping of image features a signal texture index (STI) value for the patient; and
(e) comparing the patient's STI value against a plurality of population databases, at least one database for individuals known to have already developed arthritis and a second database for individuals known to have not yet developed arthritis.

13. The method of claim 12 which can be used to prognosticate the likelihood that arthritis or damage will progress or regress in the patient.

14. The method of claim 12 wherein the arthritis to be detected is selected from the group consisting of osteoarthritis, rheumatoid arthritis, traumatic arthritis and cartilage degeneration.

15. The method of claim 13 wherein the cartilage damage to be detected is from the patient's knee, and the magnetic resonance (MR) sequences for that knee are taken for the patient's patella, medial and lateral compartments.

16. The method of claim 15 wherein the image features used to calculate the STI are taken mostly from a dominant compartment of the patient's knee where a majority of cartilage damage has occurred, said dominant compartment selected from the patient's patella, medial or lateral compartment.

17. The method of claim 12 wherein the classifier from step (b) is selected from the group consisting of a linear classifier, a non-linear classifier, a regression framework and a neural network.

18. The method of claim 12 wherein step (d) includes using one or more histogram measures selected from the group consisting of: average, mean, standard deviation, variance, dispersion, average energy, energy, skewness and kurtosis.

19. The method of claim 12 wherein step (d) includes using one or more measures selected from the group consisting of: gray level co-occurrence matrix (GLCM), gray level run length (GLRL), Z-scores, and general texture measurements.
